(19) United States
(12) Patent Application Publication
(10) Pub. No.: US 2009/0131665 A1
(43) Pub. Date: May 21, 2009

(54) Process for the preparation of crystalline forms A, B and pure crystalline form a of erlotinib HCI

(76) Inventors: Ales Gavenda, Ostrava (CZ); Jiri Faustmann, Opava (CZ); Augusto Canavesi, Locate Varesino (IT); Dietmar Flubacher, Bad Krozingen (DE)

Correspondence Address: MERCHANT & GOULD PC, P.O. BOX 2903, MINNEAPOLIS, MN, 55402-0903, US

(21) Appl. No.: 12/229,701

(22) Filed: Aug. 25, 2008

Related U.S. Application Data
(60) Provisional application No. 60/957,585, filed on Aug. 23, 2007.
(60) Provisional application No. 60/984,348, filed on Oct. 31, 2007.
(60) Provisional application No. 61/052,943, filed on May 13, 2008.
(60) Provisional application No. 61/073,990, filed on Jun. 19, 2008.
(60) Provisional application No. 60/968,207, filed on Aug. 27, 2007.
(60) Provisional application No. 61/018,160, filed on Dec. 31, 2007.
(60) Provisional application No. 61/128,658, filed on May 22, 2008.
(60) Provisional application No. 61/082,671, filed on Jul. 22, 2008.
(60) Provisional application No. 60/990,813, filed on Nov. 28, 2007.
(60) Provisional application No. 61/059,204, filed on Jun. 5, 2008.
(60) Provisional application No. 61/075,174, filed on Jun. 24, 2008.

(51) Int. Cl. C07D 239/94

(52) U.S. Cl. 544/293

(57) ABSTRACT

The invention provides processes for preparing crystalline Forms A, B and pure crystalline Form A of Erlotinib hydrochloride.

### CROSS-REFERENCE TO RELATED APPLICATIONS

This application claims the benefit of U.S. provisional application Ser. Nos. 60/957,585, filed Aug. 23, 2007; 60/984,348, filed Oct. 31, 2007; 61/052,943, filed May 13, 2008; 61/073,990, filed Jun. 19, 2008; 60/968,207, filed Aug. 27, 2007; 61/018,160, filed Dec. 31, 2007; 61/128,658, filed May 22, 2008; 61/082,671, filed Jul. 22, 2008; 60/990,813, Nov. 28, 2007; 61/059,204, Jun. 5, 2008 and 61/075,174, filed Jun. 24, 2008, each of which is incorporated herein by reference.

### FIELD OF THE INVENTION

The invention relates to processes for preparing crystalline Forms A, B and pure crystalline Form A of Erlotinib hydrochloride.

### BACKGROUND OF THE INVENTION

Erlotinib HCl, N-(3-ethynylphenyl)-6,7-bis(2-methoxyethoxy)-4-quinazolinamine hydrochloride, of the following formula

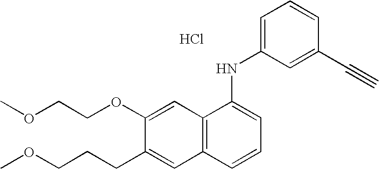

is marketed under the trade name TARCEVA® by OSI Pharmaceuticals for treatment of patients with locally advanced or metastatic non-small cell lung cancer (NSCLC) after failure of at least one prior chemotherapy regimen.

Erlotinib and its preparation are disclosed in U.S. Pat. No. 5,747,498, where the free base is produced, as shown in Scheme 1

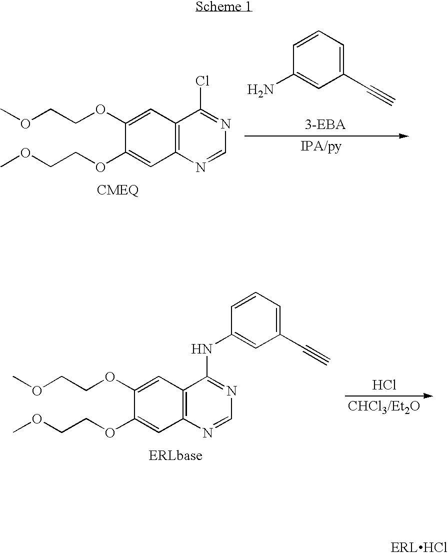

In this process, the reaction of 3-ethynylaniline (3-EBA) with 4-chloro-6,7-bis(2-methoxyethoxy)quinazoline (CMEQ) in a mixture of pyridine and isopropanol (IPA) yields the free base, which is purified by chromatography on silica gel using a mixture of acetone and hexane. The free base is then converted into the hydrochloride salt by treating a solution of ERL base in CHCl₃/Et₂O with HCl.

U.S. Pat. No. 6,476,040 discloses methods for the production of ERL and salts thereof by treatment of 4-[3-[[6,7-bis(2-methoxyethoxy]-4-quinazolinyl]amino]phenyl]-2-methyl-3-butyn-2-ol with sodium hydroxide and then with HCl in IPA, 2-methoxyethanol, 2-butanol and n-butanol) as reported in Scheme 2.

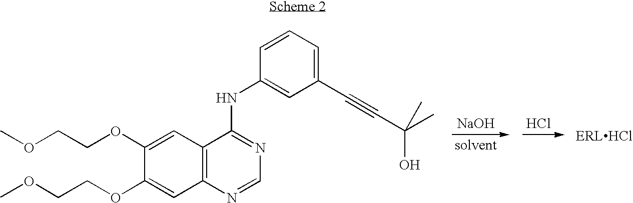

U.S. Pat. No. 6,900,221 discloses Form A that exhibits an X-ray powder diffraction pattern having characteristic peaks expressed in degrees 2-theta at approximately 5.579, 6.165, 7.522, 8.006, 8.696, 9.841, 11.251, 19.517, 21.152, 21.320, 22.360, 22.703, 23.502, 24.175, 24.594, 25.398, 26.173, 26.572, 27.080, 29.240, 30.007, 30.673, 32.759, 34.440, 36.154, 37.404 and 38.905; and Form B substantially free of Form A, wherein Form B exhibits an X-ray powder diffraction pattern having characteristics peaks expressed in degrees 2-theta at approximately 6.255, 7.860, 9.553, 11.414, 12.483, 13.385, 14.781, 15.720, 16.959, 17.668, 17.193, 18.749, 19.379, 20.196, 20.734, 21.103, 21.873, 22.452, 22.982, 23.589, 23.906, 24.459, 25.138, 25.617, 25.908, 26.527, 26.911, 27.534, 28.148, 28.617, 29.000, 29.797, 30.267, 30.900, 31.475, 31.815, 32.652, 33.245, 34.719, 35.737, 36.288, 36.809, 37.269, 37.643 and 38.114.

U.S. Pat. No. 6,900,221 also states that “the hydrochloride compound disclosed in U.S. Pat. No. 5,574,498 actually comprised a mixture of the polymorphs A and B, which because of its partially reduced stability (i.e., from the polymorph A component) was not more preferred for tablet form than the mesylate forms.”

This patent also states that the use of IPA as a solvent for preparing Form A is not recommended due to the formation of an impurity by reaction of the solvent with CMEQ.

U.S. Pat. No. 7,148,231 disclose Forms A, B, E, which are characterized by X-Ray powder diffraction, IR and melting point.

Thus, there is a need in the art for processes for preparing crystalline Forms A, B and also pure crystalline Form A of erlotinib HCl.

### SUMMARY OF THE INVENTION

One embodiment is a process for the preparation of crystalline Erlotinib hydrochloride Form A comprising reacting 4-chloro-6,7-bis(2-methoxyethoxy)quinazoline (“CMEQ”) of the following formula

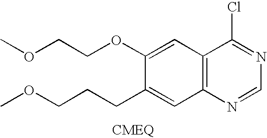

and 3-ethynylaniline (“3-EBA”) of the following formula

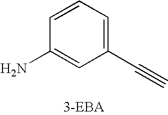

in isopropanol (“IPA”) providing a precipitate of crystalline Form A of Erlotinib HCl.

Another embodiment is a process for preparing crystalline Erlotinib hydrochloride Form A comprising crystallizing erlotinib HCl from a solvent selected from the group consisting of: toluene, a mixture of toluene and methanol, methylal, tertbutyl methylether (“TBME”), ethylacetate, n-butanol, mixture of n-butanol and water, methylisobutyl ketone (“MIBK”), s-butanol, a mixture of s-butanol and water, n-propanol, 2-propanol, methoxyethanol, a mixture of methoxyethanol and water, ethanol, a mixture of 1,3-dioxolane and methanol, a mixture of 1,3-dioxolane and water, butanone and a mixture of butanone and water; wherein the mixture of 1,3-dioxolane and water has about 2 to about 3% v/v of water, the mixture of 1,3-dioxolane and methanol has about 10% v/v of methanol, the mixture of n-butanol and water has about 1% to about 2% v/v of water, the mixture of s-butanol and water has about 1% to about 2% v/v of water, the mixture of methoxyethanol and water has about 1% to about 2% v/v of water, and the mixture of toluene and methanol has about 2% v/v of methanol.

Another embodiment is a process for the preparation of crystalline Erlotinib hydrochloride Form B comprising crystallizing erlotinib HCl from a solvent selected from the group consisting of: dichloromethane (“DCM”), diethylether, isopropyl acetate, methanol, mixture of n-butanol and water, mixture of s-butanol and water, mixture of methoxyethanol and water, mixture of 1,3-dioxolane and methanol, and mixture of 1,3-dioxolane and water, wherein the mixture of 1,3-dioxolane and water has about 5 to about 10% v/v of water, the mixture of 1,3-dioxolane and methanol has about 20% to about 40% v/v of methanol, mixture of n-butanol and water has about 5% to about 10% v/v of water, the mixture of s-butanol and water has about 10% v/v of water and the mixture of methoxyethanol and water has about 10% v/v of water.

Another embodiment is a process for the preparation of crystalline Erlotinib hydrochloride Form B comprising slurring crystalline erlotinib HCl Form A in a solvent selected from the group consisting of: methanol, mixture of 1,3-dioxolane and water, n-heptane, and diethyl ether and mixtures thereof, wherein the mixture of 1,3-dioxolane and water has about 5 to about 10% v/v of water.

### BRIEF DESCRIPTION OF THE FIGURES

FIG. 1 illustrates the powder x-ray diffraction pattern of pure crystalline Erlotinib HCl Form A.

FIG. 2 illustrates the powder x-ray diffraction pattern of Erlotinib HCl Form B.

FIG. 3 illustrates the C-13 solid-state NMR pattern of pure crystalline Erlotinib HCl Form A.

FIG. 4 illustrates C-13 solid-state NMR pattern of Erlotinib HCl Form B.

FIG. 5 illustrates dependence of filtration rate of Form A prepared under different temperatures.

FIG. 6 illustrates Microscope view of leaf-like shaped needles of Form A, prior to isolation.

FIG. 7 illustrates Microscope view of Form A of FIG. 6, after isolation-crushed particles.

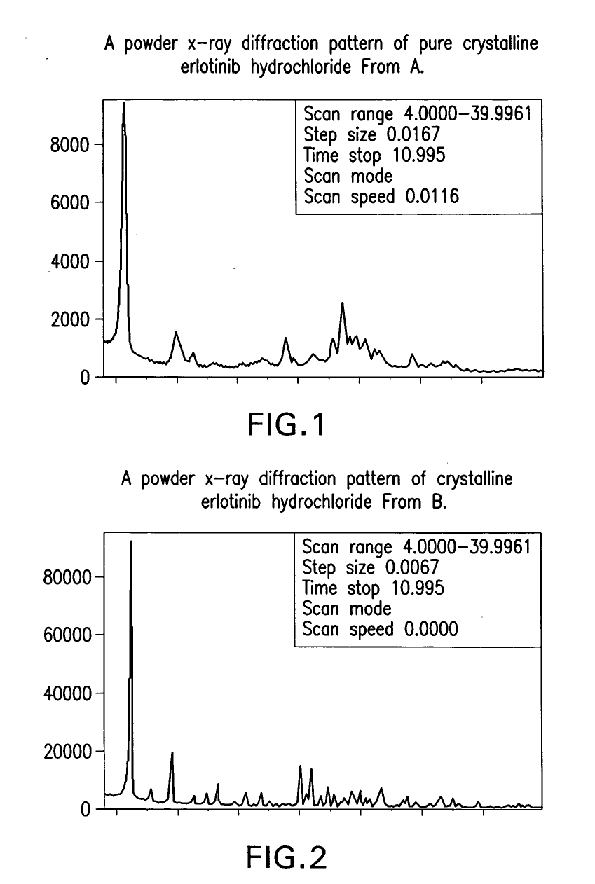

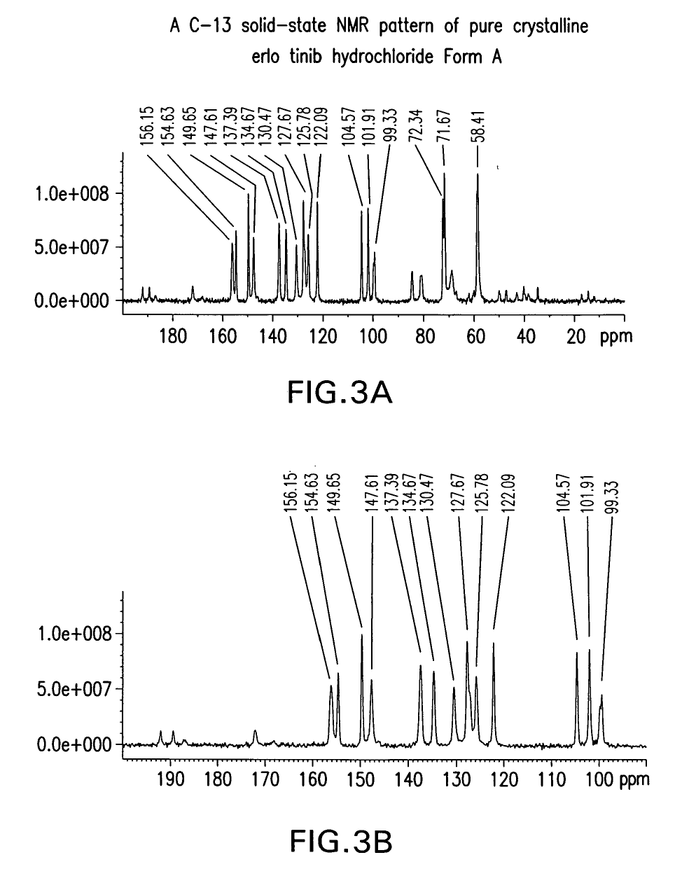

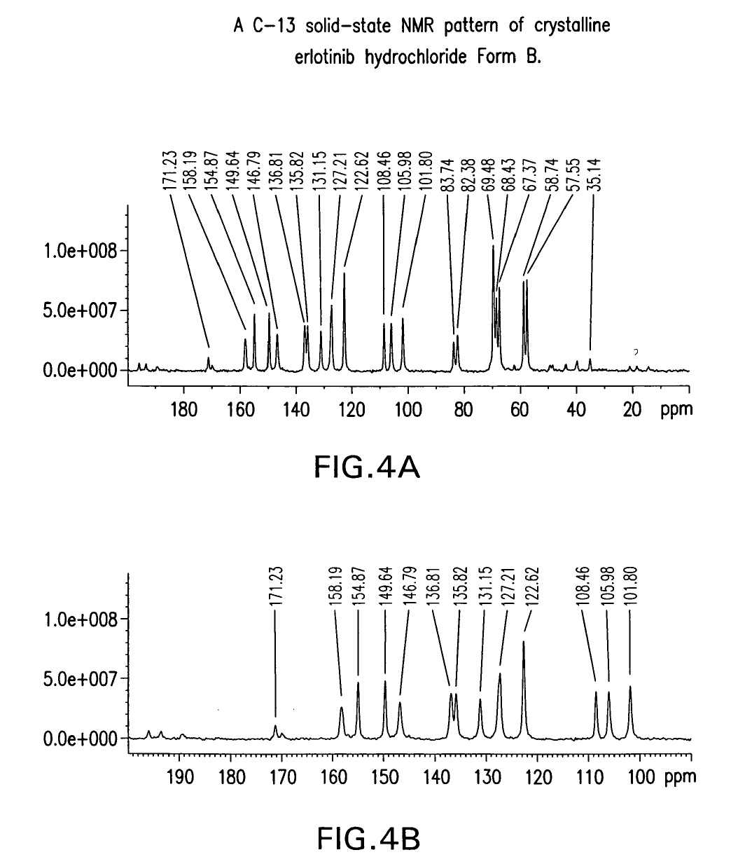

### DETAILED DESCRIPTION OF THE INVENTION

The present invention relates to processes for preparing crystalline Forms A, B and pure crystalline Form A of Erlotinib HCl.

As used herein the term “pure crystalline Form A of Erlotinib HCl” refers to crystalline Form A of erlotinib HCl exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta, containing no more than about 20% by weight of crystalline erlotinib HCl Form B, preferably not more than 10% by weight of Form B, more preferably not more than 5% by weight of Form B.

Preferably, the content of Form B provided by % by weight is measured by PXRD or by C-13 solid state NMR. When measured by PXRD, the content is determined by using one or more peaks selected from the following list of peaks 6.3, 7.8, 9.5, 12.5, 20.2 and 22.4±0.2 degrees 2-theta. More preferably XRD diffraction peak at about 6.3±0.2 degrees 2-theta. For quantification of Form B in Form A especially small percentages of Form B in Form A, the general chapter on “Characterization of crystalline solids by XRPD” of the European Pharmacopoeia 5.08, chapter 2.9.33 may be followed.

When measured by C-13 solid-state NMR, the content of form B is determined by using one or more peaks in the range 100-180 ppm selected from the following list of peaks 158.2, 136.8, 135.8, 131.2, 127.2, 122.6, 108.5, 106.0±0.2 ppm. For quantification of Form B in Form A especially small percentages of Form B in Form A, by C-13 solid-state NMR, the background can be minimized by long data collection times or other techniques known to the skilled in the art.

The first process is done by using IPA as a solvent, instead of a mixture of pyridine and IPA as described in the prior art; where the product is obtained in high yields and purity, i.e., it isn't contaminated by the said impurity.

The process for the preparation of crystalline Erlotinib hydrochloride Form A comprises reacting 4-chloro-6,7-bis(2-methoxyethoxy)quinazoline (“CMEQ”) of the following formula

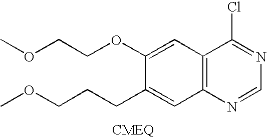

and 3-ethynylaniline (“3-EBA”) of the following formula

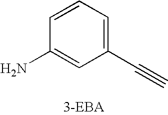

in isopropanol (“IPA”) providing a precipitate of crystalline Form A of Erlotinib HCl.

Preferably, the solvent consist of isopropanol (“IPA”).

Initially, a suspension of CMEQ and 3-EBA in IPA is heated to reflux.

Preferably, the heating is done for about 30 minutes, during which the formation of crystalline Form A of erlotinib HCl is expected to occur.

Optionally, the heated suspension can be further diluted with IPA to aid in the recovery of the crystalline form. The recovery can be done for example by filtering the suspension and drying.

Crystalline Form A can be prepared also by another process comprising crystallizing erlotinib HCl from a solvent selected from the group consisting of: toluene, a mixture of toluene and methanol, methylal, tertbutyl methylether (“TBME”), ethylacetate, n-butanol, mixture of n-butanol and water, methylisobutyl ketone (“MIBK”), s-butanol, a mixture of s-butanol and water, n-propanol, 2-propanol, methoxyethanol, mixture of methoxyethanol and water, ethanol, a mixture of 1,3-dioxolane and methanol, a mixture of 1,3-dioxolane and water, butanone and a mixture of butanone and water; wherein the mixture of 1,3-dioxolane and water has about 2 to about 3% v/v of water, the mixture of 1,3-dioxolane and methanol has about 10% v/v of methanol, the mixture of n-butanol and water has about 1% to about 2% v/v of water, the mixture of s-butanol and water has about 1% to about 2% v/v of water, the mixture of methoxyethanol and water has about 1% to about 2% v/v of water, and the mixture of toluene and methanol has about 2% v/v of methanol.

Preferably, the crystallization comprises reacting erlotinib base with HCl in the above mentioned solvents providing a suspension comprising the said crystalline Form A of erlotinib HCl.

In some embodiment, when the solvent is selected from the group consisting of: toluene, a mixture of toluene and methanol, TBME, and MIBK, the suspension is provided by combining erlotinib base and the solvent providing a first suspension; combining the first suspension with HCl to obtain a solution from which the crystalline Form A of Erlotinib HCl precipitates, providing the said suspension.

Preferably, HCl is added to the first suspension. Typically, HCl can be in a gas form or in a form of a solution. The solution can be an organic solution, such as an ether or an aqueous solution. Preferably, HCl is provided in a form of an aqueous solution. Preferably, the concentration of the aqueous solution is of about 30 to about 44% w/w, more preferably, of about 35 to about 38% w/w.

As used herein, unless defined otherwise, “w/w” refers to weight of HCl/weight of Erlotinib and “w/v” refers to weight of HCl/volume of solution. Typically, the concentration is determined by titrations with a base, as known to a skilled artisan.

Preferably, prior to the addition of HCl the temperature of the first suspension is set to about 0° C. to about 30° C.

Typically, the said suspension is maintained at the above mentioned temperature to increase the yield of the precipitated crystalline Form A and to obtain a narrower particle size distribution. Preferably, the said suspension is maintained for about 0 to about 10 hours, or about 0.5 to about 2 hours, more preferably about 1 hour.

In another preferred embodiment, when the solvent is selected from the group consisting of: methylal, ethylacetate, n-butanol, mixtures of n-butanol and water, s-butanol, a mixture of s-butanol and water, n-propanol, 2-propanol, methoxyethanol, a mixture of methoxyethanol and water, ethanol, mixtures of 1,3-dioxolane and methanol, mixtures of 1,3-dioxolane and water, butanone and mixtures of butanone and water, the suspension is provided by combining erlotinib base and the solvent providing a first solution; combining the solution with HCl to obtain the said suspension comprising crystalline Form A of Erlotinib HCl; wherein the mixture of 1,3-dioxolane and water has about 2 to about 3% v/v of water, the mixture of 1,3-dioxolane and methanol has about 10% v/v of methanol, the mixture of n-butanol and water has about 1% to about 2% v/v of water, the mixture of s-butanol and water has about 1% to about 2% v/v of water, the mixture of methoxyethanol and water has about 1% to about 2% v/v of water.

Preferably, the starting Erlotinib base can be obtained by reacting Erlotinib HCl with either an organic or inorganic base in a mixture of butanone and water.

Preferably, dissolution is achieved at about 20° C. to about 60° C. More preferably it is achieved at room temperature to about 50° C.

Preferably, HCl is added to the first solution. Typically, HCl can be in a gas form or in a form of a solution. The solution can be an organic solution, such as an ether or an aqueous solution. Preferably, HCl is provided in a form of an aqueous solution. Preferably, the concentration of the aqueous solution is of about 30 to about 44% w/w, more preferably, of about 35 to about 38% w/w. Typically, the concentration is determined by titrations with a base, as known to a skilled artisan.

The above process can also lead to pure Form A having a large particle size; by performing the precipitation of crystalline Form A at a temperature of about 0° C. to about 75° C. In more preferred embodiments, when the solvents is a mixture of 1,3-dioxalane having about 2% to about 3% of water v/v or 1,3-dioxalane having about 10% of methanol, the temperature is set to about 20° C. to about 75° C., more preferably, to a temperature of about 60° C. to about 75° C., most preferably, to a temperature of about 60° C. to about 70° C., and these conditions lead to crystalline Form A that has large particle size. As used herein, unless defined otherwise, the term “large” when referring to the particle size of crystalline Erlotinib HCl Form A means that the majority of particles are between about one hundred to several hundred microns long. For example, a typical population of large particles might have a D(90) of about 300 microns. This is advantageous when recovering the said crystalline form due to enhanced filterability as exemplified in Example 3.

Crystalline erlotinib HCl Form A having such a size can be prepared by employing a warm crystallization, e.g., about 50° C. to about 75° C., preferably, about 60° C. to about 70° C., most preferably about 60° C. This is advantageous when recovering the crystalline form due to enhanced filterability, for example, as shown in Example 3.

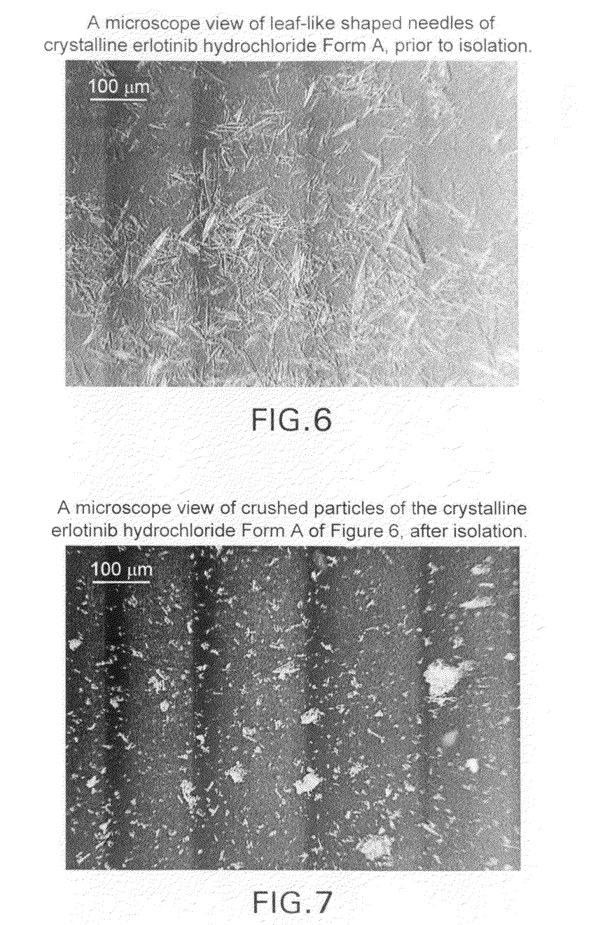

Also, the crystal shape of ERL hydrochloride Form A is usually leaf-like needles, as demonstrated by FIGS. 6 and 7. These crystals are very fragile so in the course of isolation (filtration and drying) they are able to break down into much smaller fragments. Thus, generally, suspensions of Form A exhibit very poor filtration properties, which could cause difficulties in large-scale production, as exemplified in Example 3. Therefore, it would be desirable to develop a process which enables the preparation of Form A or pure form A having better filterability and enabling preparation/isolation thereof.

In a most preferred embodiment, the crystallization from a mixture of 1,3-dioxolane having about 2% to about 3% of water v/v and 1,3-dioxolane having about 10% of methanol provides pure crystalline form A of erlotinib HCl.

Typically, the temperature of the suspension can be decreased to increase the yield of the precipitated crystalline Erlotinib HCl Form A. Preferably, the temperature can be decreased to about 40° C. to about 0° C., more preferably, to about 40° C. to about 25° C. The suspension can then be further maintained. Preferably, the suspension can then be further maintained for about 1 hour to about 24 hours, more preferably, for about 4 to about 12 hours.

The process for preparing crystalline Form A can further comprise a recovery process. The recovery can be done, for example by filtering the suspension and drying.

In yet other preferred embodiment, when the solvent is methoxyethanol, the crystallization can be done by using erlotinib HCl as a starting material, instead of Erlotinib base. The process comprises dissolving erlotinib HCl in methoxyethanol, and precipitating to obtain the said suspension comprising precipitated crystalline Form A of erlotinib HCl.

Preferably, dissolution is achieved at a temperature of about 98° C. to about 92° C., more preferably at about 95° C.

Preferably, precipitation is done by cooling the solution to a temperature of about +10° to about −10° C., more preferably to about 0° C. Optionally, seeding of crystalline Form A can be done to aid in precipitation of the product. Typically, the cooling provides the said suspension comprising precipitated crystalline erlotinib HCl Form A.

Further, the said suspension can be further maintained at the above temperature to increase the yield of the precipitated crystalline Form A and to obtain a higher yield and a narrower particle size distribution. Preferably, the suspension is maintained for about 0 hours to about 2 hours, more preferably for about 2 hours. To increase the yield even more, the cooled suspension can be further maintained for about 15 hours to about 24 hours, more preferably for about 15 hours, at a temperature of about −10° C. to about −40° C., more preferably at about −15° C. to about −25° C., most preferably at about −20° C.

The precipitated crystalline Form A can then be recovered for example, by using a centrifuge.

Another embodiment of the invention is a process for the preparation of crystalline Erlotinib hydrochloride Form B. The process comprises crystallizing Erlotinib HCl from a solvent selected from the group consisting of: dichloromethane (“DCM”), diethylether, isopropyl acetate, methanol, mixture of n-butanol and water, mixture of s-butanol and water, mixture of methoxyethanol and water, mixture of 1,3-dioxolane and methanol, mixture of 1,3-dioxolane and water, wherein the mixture of 1,3-dioxolane and water has about 5 to about 10% v/v of water, the mixture of 1,3-dioxolane and methanol has about 20% to about 40% v/v of methanol, mixture of n-butanol and water has about 5% to about 10% v/v of water, the mixture of s-butanol and water has about 10% v/v of water and the mixture of methoxyethanol and water has about 10% v/v of water.

Preferably, the crystallization comprises reacting erlotinib base with HCl in the above mentioned solvents and precipitating to obtain the suspension comprising of the said crystalline Form B of erlotinib HCl.

Initially, erlotinib base is dissolved in the solvent. Preferably, dissolution is achieved at about 20° C. to about 60° C., more preferably at about room temperature. Then, the solution of erlotinib base is reacted with HCl. Preferably, HCl is added to the solution. Typically, HCl can be in a gas form or in a form of a solution. The solution can be an organic solution, such as an ether or an aqueous solution. Preferably, the ether is diethyl ether. Preferably, HCl is provided in a form of an aqueous solution. Preferably, the concentration of the aqueous solution is of about 30 to about 44% w/w, more preferably, of about 35 to about 38% w/w. Typically, the concentration is determined by titrations with a base, as known to a skilled artisan.

Preferably, prior to the addition of HCl the temperature of the solution is set to about 0° C. to about 60° C., more preferably at about 30° C. to about 60° C., most preferably, at about 30° C.

Typically, the said suspension is maintained at the above mentioned temperature to increase the yield of the precipitated crystalline Form B. Preferably, the said suspension is maintained for about 1 hour to 24 hours, more preferably for about 1 hour.

The process for preparing crystalline Form B can further comprise a recovery process. The recovery can be done, for example by filtering the suspension and drying.

In a preferred embodiment, when the solvent is methanol the crystallization can be done by using erlotinib HCl as a starting material, instead of Erlotinib base. The process comprises dissolving erlotinib HCl in methanol, and precipitating to obtain the said suspension comprising of precipitated crystalline Form B of erlotinib HCl.

Preferably, dissolution is achieved at a temperature of about 65° C.

Preferably, precipitation is done by cooling the solution to a temperature of about +10° to −10° C., more preferably to about 0° C. Optionally, seeding of crystalline Form B can be done to aid in precipitation of the product. Typically, the cooling provides the said suspension comprising of precipitated crystalline erlotinib HCl Form B.

Further, the said suspension can be further maintained at the above temperature to increase the yield of the precipitated crystalline Form B and to obtain a narrower particle size distribution. Preferably, the suspension is maintained for about 0 hours to about 24 hours, more preferably for about 1 hour to about 4 hours, most preferably, for about 2 hours. To increase the yield even more, the cooled suspension can be further maintained for 15 hours to about 24 hours, more preferably for about 15 hours, at a temperature of about −10° to about −40° C., more preferably at about −15° C. to about −25° C., most preferably at about −20° C.

The precipitated crystalline Form B can then be recovered for example, by filtration and drying.

Crystalline Form B can be prepared also by another process comprising slurrying crystalline erlotinib HCl Form A in a solvent selected from the group consisting of: methanol, mixture of 1,3-dioxolane and water, n-heptane, and diethyl ether, wherein the mixture of 1,3-dioxolane and water has about 5 to about 10% v/v of water and mixtures thereof.

Preferably, slurrying is done at a temperature of about 0° C. to about 30° C.

The process for preparing crystalline Form B can further comprise recovering it from the slurry. The recovery can be done, for example by filtering the slurry and drying.

### EXAMPLES

### PXRD

XRD diffraction was performed on X-Ray powder diffractometer: Philips X'pert Pro powder diffractometer equipped with X'celerator multichannel detector, detector active length 2.122 mm., Cu-tube, CuKα radiation, λ=1.541874 Å. A stainless steel sample holder with zero background silicon plate. Scanning parameters: Range 4-40 degrees two-theta; Continuous scan; Step size 0.0167 deg; Scan rate 6 deg./min. Prior to analysis the samples were gently ground by means of mortar and pestle in order to obtain a fine powder. The ground sample was adjusted into a cavity of the sample holder and the surface of the sample was smoothed by means of a microscopic glass slide.

### Solid-State NMR

Bruker Avance 500 WB/US NMR spectrometer (Karlsruhe, Germany, 2003). 125 MHz, Magic angle spinning (MAS) frequency 11 kHz, 4 mm ZrO2 rotors and standard CPMAS pulse program was used.

### Microscope

An optical microscope system with polarized light, CCD camera and datasoftware.

### Example 1

### Preparation of Crystalline Form A of Erlotinib HCl

A suspension of CMEQ (48.0 g; 153 mmol) and 3-EBA (19.7 g; 168 mmol) in IPA (1000 mL) was mechanically stirred under reflux for 30 min. The resulting thick suspension was diluted with EPA (500 mL) and the precipitate was collected, washed with IPA and dried under vacuum at 40° C. to give ERL HCl Form A as a colorless solid (63.8 g; 97% yield).

### Example 2

### Preparation of Pure Crystalline Form A of Erlotinib HCl

Erlotinib base (waterless, 2.00 g, 5.083 mmole) was dissolved in water-1,3-dioxolane mixture (80 ml). The content of water was adjusted at 2-3% v/v. Temperature of the solution was adjusted at certain value—it may range from 0° C. to 75° C. 414 μl (mole/mole) of concentrated hydrochloric acid (44.1% w/v) (concentration determined by titrations) was added slowly (during 10 min) into solution. Solid phase was created immediately. The crystalline suspension was agitated for 1 hr while keeping the selected temperature and then cooled to 0° C. The crystalline phase was separated by filtration, rinsed with 2% water-1,3-dioxolane mixture (40 ml) and dried on the filter by blowing nitrogen through the cake to the constant weight. The drying was finished in a small laboratory oven under nitrogen ventilation at 40° C. for 3 hrs. Erlotinib hydrochloride Form A was obtained (molar yield about 95%).

### Example 3

### Preparation of Crystalline Erlotinib HCl Form A with Improved Filterability

Erlotinib base (waterless, 2.00 g, 5.083 mmole) was dissolved in water-1,3-dioxolane mixture (80 ml). The content of water was adjusted at 2% v/v. Temperature of the solution was set up to 60° C. 414 μl (mole/mole) of concentrated hydrochloric acid (44.1% w/v) was added slowly (during 10 min) into solution. Solid phase was created immediately. The crystalline suspension was agitated for 1 hr while keeping the selected temperature (60° C.) and then cooled to 40° C. The suspension was agitated for 24 hrs while keeping the temperature at 40° C. After carrying out granulation the crystalline phase was separated by filtration, rinsed with 2% water-1,3-dioxolane mixture (40 ml) and dried on the filter by blowing nitrogen through the cake to the constant weight. The drying was finished in small laboratory oven under nitrogen ventilation at 40° C. for 3 hrs.

Erlotinib hydrochloride Form A was obtained (2.13 g, yield 97.5%).
The filtration parameters of suspension:
a=26 122 sm⁻²
b=27 sm⁻¹
   (parameters are valid for overpressure 100 kPa, measured at comparable conditions)

### Example 4

### The Correlation of Granulation Temperature and Filterability

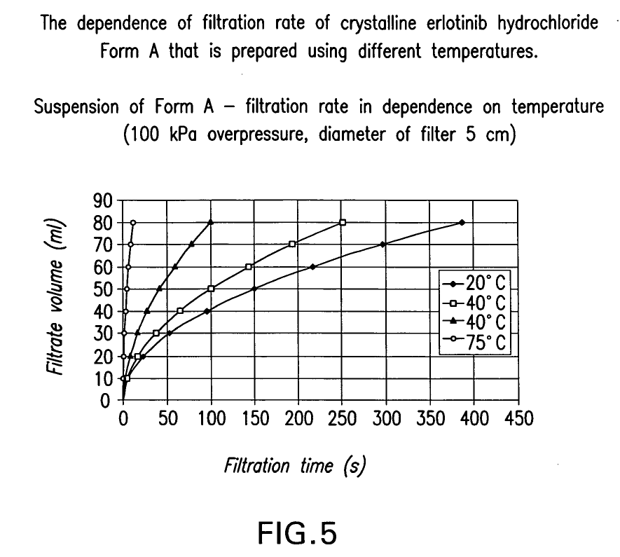

Temperature during precipitation and granulation strongly influences filterability of crystalline Form A suspension. Filtration properties are getting better with increasing precipitation and granulation temperature. Filtration tests with suspensions precipitated at various temperatures have been performed. The results are presented in the following table and graph (see FIG. 5) illustrating dependence of filtration rate on crystallization temperature.

### Example 5

### Preparation of Crystalline Form A of Erlotinib HCl

Erlotinib base (3 g) was added to a mixture of dioxolane (78.4 mL) and water (1.6 mL) and the temperature of the solution was adjusted to 60° C. At this temperature conc. HCl (7.63 mmol) was added. Precipitation occurred immediately. The suspension was stirred for 1 h at 60° C., then cooled to 0° C. The solid was filtered off and dried at 110° C. under N₂ ventilation for 4 h. Crystalline Form A of ERL HCl was obtained with 95% yield.

### Example 6

### Preparation of Crystalline Form A of Erlotinib HCl

Erlotinib hydrochloride (500 mg), was dissolved in methoxyethanol (35 mL) by heating at 95° C. until a complete solution was obtained. The process was performed on rotary evaporator. The bulb was cooled to 0° C. and supersaturated solution was seeded by a negligible amount of Form A. The crystalline suspension was agitated on a rotary evaporator for 2 h at 0° C. and then let to stay into a freezer overnight. In the morning the crystalline phase was separated by sedimentation centrifuge and the sediment was taken up with 1,3-dioxolane (20 mL). The solid was filtered off and dried under nitrogen stream at room temperature. Crystalline Form A of Erlotinib hydrochloride was obtained (353 mg, yield 70.6%).

### Example 7

### Preparation of Crystalline Form A of Erlotinib HCl

1 g of Erlotinib was dissolved in a mixture of 20 g butanone and 2 g of water at 50° C., under stirring 0.3 g of aqueous 37% hydrochloric acid solution was added obtaining immediate precipitation. After half an hour at room temperature the suspension was filtered on Buckner filter. The precipitate was rinsed with butanone and dried at 60° under vacuum for one hour obtaining 0.9 g of Erlotinib hydrochloride.

### Example 8

### Preparation of Crystalline Form A of Erlotinib HCl

10 g of Erlotinib hydrochloride were dissolved in a mixture of 200 g of butanone, 30 g of water, and 5 g of aqueous ammonia 27%. The organic phase was separated and washed 2 times with 30 g of water. The solution was concentrated, eliminating 20 ml of distillate, and 20 ml of fresh butanone were added to adjust the original volume.

Under stirring 3 g of aqueous 37% hydrochloric acid solution were added obtaining rapidly crystallization. After one hour under stirring at room temperature the suspension was filtered, the cake was rinsed with butanone and the product was dried overnight at 60° under vacuum. 9 g of Erlotinib hydrochloride were obtained.

### Example 9

### Preparation of Crystalline Form A of Erlotinib HCl

### General Procedure:

Erlotinib base (anhydrous), one weight portion was dissolved at RT in 40 volume portions of solvent or solvent mixture listed in the table. Temperature of the solution was adjusted at given value (in the table). 207 μl per one gram of the base (equivalent amount) of concentrated hydrochloric acid*) was added into solution. New crystalline phase was created immediately or during one minute. The crystalline suspension was agitated for 1 hr holding the above selected temperature and then cooled to 0° C. The solid was separated by filtration or centrifugation and dried in the nitrogen stream to the constant weight. Some batches were dried in a small laboratory oven under nitrogen ventilation. The drying conditions are listed in the table.

*) HCl content determination was performed by titration: 44.1% w/v

TABLE 1
Crystallization Conditions Leading to Form A

| | | | | Precipitation and | | | | | |
|---|---|---|---|---|---|---|---|---|---|
| Solvent mixture used (40 volume | | Charge of erlotinib | | granulation | | | | | |
| portions of base) | | base | | temperature | | Crystalline Form | Molar yield | Drying conditions | |
| Solvent I | Solvent II | [mg] | | [° C.] | | obtained | [%] | [%] | |
| Toluene | | 50 | mg *) | 30° | C. | Form A | — | Temperature | RT |
| 100% v/v | | | | | | | | | nitrogen ventilation |
| Toluene | Methanol | 50 | mg *) | 30° | C. | Form A | — | Temperature | RT |
| 98% v/v | 2% v/v | | | | | | | | nitrogen ventilation |
| Methylal | | 50 | mg | 0° | C. | Form A | — | Temperature | RT |
| 100% v/v | | | | | | | | | nitrogen ventilation |
| TBME | | 50 | mg *) | 0° | C. | Form A | — | Temperature | RT |
| 100% v/v | | | | | | | | | nitrogen ventilation |
| Ethylacetate | | 50 | mg | 0° | C. | Form A | — | Temperature | RT |
| 100% v/v | | | | | | | | | nitrogen ventilation |
| n-buthanol | | 50 | mg | 30° | C. | Form A | — | Temperature | RT |
| 100% v/v | | | | | | | | | nitrogen ventilation |
| n-buthanol | Water | 50 | mg | 30° | C. | Form A | — | Temperature | RT |
| 99% v/v | 1% v/v | | | | | | | | nitrogen ventilation |
| n-buthanol | Water | 50 | mg | 30° | C. | Form A | — | Temperature | RT |
| 98% v/v | 2% v/v | | | | | | | | nitrogen ventilation |
| MIBK | | 50 | mg *) | 0° | C. | Form A | — | Temperature | RT |
| 100% v/v | | | | | | | | | nitrogen ventilation |
| s-buthanol | | 50 | mg | 30° | C. | Form A | — | Temperature | RT |
| 100% v/v | | | | | | | | | nitrogen ventilation |
| s-buthanol | Water | 50 | mg | 30° | C. | Form A | — | Temperature | RT |
| 99% v/v | 1% v/v | | | | | | | | nitrogen ventilation |
| s-buthanol | Water | 50 | mg | 30° | C. | Form A | — | Temperature | RT |
| 98% v/v | 2% v/v | | | | | | | | nitrogen ventilation |
| n-propanol | | 50 | mg | 0° | C. | Form A + | — | Temperature | RT |
| 100% v/v | | | | | | traces B | | | nitrogen ventilation |
| 2-propanol | | 50 | mg | 0° | C. | Form A + | — | Temperature | RT |
| 100% v/v | | | | | | traces B | | | nitrogen ventilation |
| Methoxyethanol | | 50 | mg | 30° | C. | Form A | — | Temperature | RT |
| 100% v/v | | | | | | | | | nftrogen ventilation |
| Methoxyethanol | Water | 50 | mg | 30° | C. | Form A | — | Temperature | RT |
| 99% v/v | 1% v/v | | | | | | | | nitrogen ventilation |
| Methoxyethanol | Water | 50 | mg | 30° | C. | Form A | — | Temperature | RT |
| 98% v/v | 2% v/v | | | | | | | | nitrogen ventilation |
| Ethanol | | 50 | mg | 0° | C. | Form A | — | Temperature | RT |
| 100% v/v | | | | | | | | | nitrogen ventilation |
| 1..3-Dioxolane | Methanol | 50 | mg | 60° | C. | Form A | — | Temperature | RT |
| 90% v/v | 10% v/v | | | | | | | | nitrogen ventilation |
| 1..3-Dioxolane | Water | 50 | mg | 60° | C. | Form A | — | Temperature | RT |
| 98% v/v | 2% v/v | | | | | | | | nitrogen ventilation |
| 1..3-Dioxolane | Water | 50 | mg | 60° | C. | Form A | — | Temperature | RT |
| 97% v/v | 3% v/v | | | | | | | | nitrogen ventilation |

*) only partial dissolution of the base at given conditions, completely dissolution of starting material was achieved after addition of HCl.

TABLE 2
Crystallization Conditions Leading to Form A

| | | | Precipitation and | | | | |
|---|---|---|---|---|---|---|---|
| Solvent mixture used (40 volume | | Charge of erlotinib | granulation | | | | |
| portions of base) | | base | temperature | Crystalline Form | Molar yield | Drying conditions | |
| Solvent I | Solvent II | [mg] | [° C.] | obtained | [%] | [%] | |
| 1..3-Dioxolane | Water | 2000 mg | 20° C. | Form A | 96.6% | Temperature | 40° c. |
| 98% v/v | 2% v/v | | | | | 3 hrs | nitrogen ventilation |
| 1..3-Dioxolane | Water | 2000 mg | 40° C. | Form A | 92.0% | Temperature | 40° c. |
| 98% v/v | 2% v/v | | | | | 3 hrs | nitrogen ventilation |
| 1..3-Dioxolane | Water | 2000 mg | 60° C. | Form A | 87.9% | Temperature | 40° c. |
| 98% v/v | 2% v/v | | | | | 3 hrs | nitrogen ventilation |
| 1..3-Dioxolane | Water | 2000 mg | 75° C. | Form A | 76.0% | Temperature | 40° c. |
| 98% v/v | 2% v/v | | | | | 3 hrs | nitrogen ventilation |
| 1..3-Dioxolane | Water | 3000 mg | 60° C. | Form A | 94.90% | Temperature | 110° c. |
| 98% v/v | 2% v/v | | | | | 4 hrs | nitrogen ventilation |

### Example 10

### Preparation of Crystalline Form B of Erlotinib HCl-Crystallization Processes

### General Procedure:

Erlotinib base (anhydrous), one weight portion was dissolved at RT in 40 volume portions of solvent or solvent mixture listed in the table. Temperature of the solution was adjusted at given value (in the table). 207 μl per one gram of the base (equivalent amount) of concentrated hydrochloric acid*) was added into solution. New crystalline phase was created immediately or during one minute. The crystalline suspension was agitated for 1 hr holding the above selected temperature and then cooled to 0° C. The solid was separated by filtration or centrifugation and dried in the nitrogen stream to the constant weight.

*) HCl content determination was performed by titration: 44.1% w/v

### Table 3

### Crystallization Conditions Leading to Form B (Crystallization Processes)

TABLE 3
Crystallization Conditions Leading to Form B (crystallization processes)

| | | | Precipitation and | | | | |
|---|---|---|---|---|---|---|---|
| Solvent mixture used (40 volume | | Charge of erlotinib | granulation | | | | |
| portions of base) | | base | temperature | Crystalline Form | Molar yield | Drying conditions | |
| Solvent I | Solvent II | [mg] | [° C.] | obtained | [%] | [%] | |
| Dichlormethane | | 50 mg | 0° C. | Form B | — | Temperature | RT |
| 100% v/v | | | | | | | nitrogen ventilation |
| Diethylether | | 50 mg | 0° C. | Form B | — | Temperature | RT |
| 100% v/v | | | | | | | nitrogen ventilation |
| Isopropylacetat | | 50 mg | 0° C. | Form B | — | Temperature | RT |
| 100% v/v | | | | | | | nitrogen ventilation |
| Methanol | | 50 mg | 0° C. | Form B | — | Temperature | RT |
| 100% v/v | | | | | | | nitrogen ventilation |
| n-buthanol | Water | 50 mg | 30° C. | Form B | — | Temperature | RT |
| 95% v/v | 5% v/v | | | | | | nitrogen ventilation |
| n-buthanol | Water | 50 mg | 30° C. | Form B | — | Temperature | RT |
| 90% v/v | 10% v/v | | | | | | nitrogen ventilation |
| s-buthanol | Water | 50 mg | 30° C. | Form B | — | Temperature | RT |
| 90% v/v | 10% v/v | | | | | | nitrogen ventilation |
| Methoxyethanol | Water | 50 mg | 30° C. | Form B | — | Temperature | RT |
| 90% v/v | 10% v/v | | | | | | nitrogen ventilation |
| 1..3-Dioxolane | Methanol | 50 mg | 60° C. | Form B | — | Temperature | RT |
| 80% v/v | 20% v/v | | | | | | nitoogen ventilation |
| 1..3-Dioxolane | Methanol | 50 mg | 60° C. | Form B | — | Temperature | RT |
| 70% v/v | 30% v/v | | | | | | nitrogen ventilation |
| 1..3-Dioxolane | Methanol | 50 mg | 60° C. | Form B | — | Temperature | RT |
| 60% v/v | 40% v/v | | | | | | nitrogen ventilation |
| 1..3-Dioxolane | Water | 50 mg | 60° C. | Form B | — | Temperature | RT |
| 95% v/v | 5% v/v | | | | | | nitrogen ventilation |
| 1..3-Dioxolane | Water | 50 mg | 60° C. | Form B | — | Temperature | RT |
| 90% v/v | 10% v/v | | | | | | nitrogen ventilation |
| 1..3-Dioxolane | Water | 500 mg | 60° C. | Form B | 61% | Temperature | RT |
| 90% v/v | 10% v/v | | | | | | nitrogen ventilation |

### Example 11

### Preparation of Crystalline Form B of Erlotinib HCl-Slurry Processes

### General Procedure:

Erlotinib hydrochloride Form A was placed into glass vial together with certain amount of solvent or solvents mixture (real volumes are listed in the table). Temperature of the solution was adjusted at given value (listed in the table) and the content of vial was agitated with magnetic stirrer holding the temperature for 4 hrs. The solid was separated by filtration and dried on the filter by blowing nitrogen through the cake at RT.

TABLE 4
Crystallization Conditions Leading to Form B (slurry processes)

| | | Charge of erlotinib | | Granulation | | | | |
|---|---|---|---|---|---|---|---|---|
| Solvent mixture used | | HCl (Form A) | Volume of solvent | temperature | | Crystalline Form | Drying conditions | |
| Solvent I | Solvent II | [mg] | [ml] | [° C.] | | obtained | [%] | |
| n-heptane | | 10 mg | 1 ml | 0° | C. | Form B | Temperature | RT |
| 100% v/v | | 10 mg | 1 ml | 20° | C. | Form B | | nitrogen ventilation |
| Dielhylether | | 10 mg | 1 ml | 0° | C. | Form B | Temperature | RT |
| 100% v/v | | 10 mg | 1 ml | 20° | C. | Form B | | nitrogen ventilation |
| Methanol | | 10 mg | 1 ml | 0° | C. | Form B | Temperature | RT |
| 100% v/v | | 10 mg | 1 ml | 20° | C. | Form B | | nitrogen ventilation |
| Dioxolane | water | 50 mg | 2 ml | 30° | C. | Form B | Temperature | RT |
| 95% v/v | 5% v/v | | | | | | | nitrogen ventilation |
| Dioxolane | water | 50 mg | 2 ml | 30° | C. | Form B | Temperature | RT |
| 90% v/v | 10% v/v | | | | | | | nitrogen ventilation |

### Example 12

### Preparation of Crystalline Form B of Erlotinib HCl

Erlotinib base (500 mg) was added to a mixture of 1,3-dioxolane (18 mL) and water (2 mL) and the temperature of the solution was adjusted to 60° C. At this temperature conc. HCl (1.27 mmol) was added. Precipitation occurred immediately. The suspension was stirred for 1 h at 60° C., and then cooled to 0° C. The solid was filtered off and dried under nitrogen stream at room temperature. Crystalline Form B of Erlotinib hydrochloride was obtained with 61% yield.

### Example 13

### Preparation of Crystalline Form B of Erlotinib HCl

Erlotinib hydrochloride (500 mg) was dissolved in methanol (50 mL) by heating at reflux (65° C.) until a complete solution was obtained. The whole process was performed on rotary evaporator. The bulb was cooled to 0° C. and supersaturated solution was agitated on rotary evaporator for 2 h at 0° C. and then let to stay into freezer overnight. The solid was separated by filtration and dried in nitrogen stream at room temperature. Crystalline Form B of Erlotinib hydrochloride was obtained (420 mg, yield 76.9%)

### Example 14

### Preparation of Crystalline Form B of Erlotinib HCl

In a glass vial, Erlotinib hydrochloride Form A (10 mg) was suspended into Et₂O, the suspension was cooled at 0° C. and stirred at this temperature for 4 h. The solid was separated by filtration and dried on the filter by blowing nitrogen through the cake. Erlotinib hydrochloride Form B was obtained.

What is claimed is:

1. A process for the preparation of crystalline form of erlotinib hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta, comprising reacting 4-chloro-6,7-bis(2-methoxyethoxy)quinazoline (“CMEQ”) of the following formula 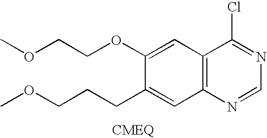
and 3-ethynylaniline (“3-EBA”) of the following formula 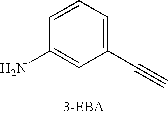
in isopropanol (“IPA”) to produce the said crystalline form of erlotinib hydrochloride.

2. The process of claim 1, wherein said process comprises heating a suspension of said CMEQ and 3-EBA in IPA to reflux.

3. The process of any one of claims 1 or 2, further comprising recovering the said crystalline form of erlotinib hydrochloride from the said suspension.

4. A process for preparing crystalline form of erlotinib hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta, comprising crystallizing erlotinib hydrochloride from a solvent selected from the group consisting of: toluene, a mixture of toluene and methanol, methylal, tertbutyl methyl ether (“TBME”), ethyl acetate, n-butanol, mixture of n-butanol and water, methylisobutyl ketone (“MIBK”), s-butanol, a mixture of s-butanol and water, n-propanol, 2-propanol, methoxyethanol, mixture of methoxyethanol and water, ethanol, a mixture of 1,3-dioxolane and methanol, a mixture of 1,3-dioxolane and water, butanone and a mixture of butanone and water to produce a suspension comprising said crystalline form of erlotinib, hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta; wherein the mixture of 1,3-dioxolane and water has about 2 to about 3% v/v of water, the mixture of 1,3-dioxolane and methanol has about 10% v/v of methanol, the mixture of n-butanol and water has about 1% to about 2% v/v of water, the mixture of s-butanol and water has about 1% to about 2% v/v of water, the mixture of methoxyethanol and water has about 1% to about 2% v/v of water, and the mixture of toluene and methanol has about 2% v/v of methanol.

5. The process of claim 4, wherein erlotinib HCl is made by reacting erlotinib base with hydrochloric acid in said solvent.

6. The process of claim 4 or claim 5, wherein said solvent is selected from the group consisting of: toluene, a mixture of toluene and methanol, TBME, and MIBK, and wherein said suspension is produced by a method comprising (i) combining erlotinib base and said solvent to produce a first suspension; and (ii) combining said first suspension with HCl to precipitate said crystalline form of erlotinib HCl.

7. The process of claim 5 or claim 6, wherein said HCl is in a gas form or in a form of a solution.

8. The process of claim 7, wherein said HCl is dissolved in an organic solvent.

9. The process of claim 8, wherein the organic solvent is ether, preferably diethylether.

10. The process of claim 7, wherein said HCl is in a form of an aqueous solution.

11. The process of claim 10, wherein concentration of the HCl is about 30 to about 44% w/w.

12. The process of any one of claims 5 to 11, wherein, prior to the addition of said hydrochloride, the temperature of the first suspension is set to about 0° C. to about 30° C.

13. The process of claim 4 or claim 5, wherein said solvent is selected from the group consisting of: methylal, ethylacetate, n-butanol, mixtures of n-butanol and water, s-butanol, a mixture of s-butanol and water, n-propanol, 2-propanol, methoxyethanol, a mixture of methoxyethanol and water, ethanol, mixtures of 1,3-dioxolane and methanol, mixtures of 1,3-dioxolane and water, butanone and mixtures of butanone and water, and wherein said suspension is produced by (i) combining erlotinib base and said solvent to produce a first solution; and (ii) combining said solution with hydrochloride to obtain said suspension comprising crystalline form of erlotinib hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta; wherein the mixture of 1,3-dioxolane and water has about 2 to about 3% v/v of water, the mixture of 1,3-dioxolane and methanol has about 10% v/v of methanol, the mixture of n-butanol and water has about 1% to about 2% v/v of water, the mixture of s-butanol and water has about 1% to about 2% v/v of water, the mixture of methoxyethanol and water has about 1% to about 2% v/v of water.

14. The process of claim 13, wherein erlotinib base is obtained by reacting erlotinib hydrochloride with either an organic or inorganic base in a mixture of butanone and water.

15. The process of any of claims 13-14, wherein step (i) is carried out at about 20° C. to about 60° C.

16. The process of any of claims 13-15, wherein said HCl is in a gas form or in a form of a solution.

17. The process of claim 16, wherein said HCl is dissolved in an organic solvent.

18. The process of claim 17, wherein said organic solvent is an ether, preferably diethylether.

19. The process of claim 16, wherein said HCl is in a form of an aqueous solution.

20. The process of claim 19, wherein concentration of said aqueous solution is about 30 to about 44% w/w.

21. The process of any one of claims 13 to 20, wherein prior to the addition of HCl, the temperature of the solution is set to about 0° C. to about 75° C.

22. The process of claim 21, wherein said solvent is a mixture of 1,3-dioxolane having about 2% to about 3% of water v/v or 1,3-dioxolane having about 10% of methanol.

23. The process of claim 22, wherein the temperature of the solution is set to about 20° C. to about 75° C.

24. The process of claim 22, wherein the temperature of the solution is set to about 60° C. to about 70° C.

25. The process of any one of claims 4 to 20, further comprising recovering the said crystalline form of erlotinib hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta.

26. The process of any of claims 4-25, wherein the obtained crystalline form of erlotinib hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta, having about 90% of the particles have a size of 300 μm or a little less.

27. The process of any of claims 4-5, wherein the solvent is methoxyethanol, and wherein said process comprises (a) dissolving erlotinib HCl in methoxyethanol, and (b) precipitating to obtain the said suspension comprising said crystalline form of erlotinib hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta.

28. The process of claim 27, wherein said dissolving is carried out at a temperature of about 92° C. to about 98° C.

29. The process of claim 28, wherein said precipitating is carried out by cooling the solution to a temperature of about +10° to about −10° C.

30. The process of any of claims 27-29, wherein the suspension is further maintained for about 15 hours to about 24 hours at a temperature of about −10° C. to about −40° C.

31. The process of any of claims 27-30, wherein said crystalline form of erlotinib hydrochloride, is recovered by centrifugation.

32. A process for the preparation of crystalline form of erlotinib hydrochloride exhibiting an X-ray powder diffraction pattern having characteristics peaks at approximately 6.3, 7.8, 9.5, 12.5, 20.2 and 22.4±0.2 degrees 2-theta. More preferably XRD diffraction peak at about 6.3±0.2 degrees 2-theta, comprising crystallizing erlotinib hydrochloride from a solvent selected from a group consisting of dichloromethane (“DCM”), diethylether, isopropyl acetate, methanol, mixture of n-butanol and water, mixture of s-butanol and water, mixture of methoxyethanol and water, mixture of 1,3-dioxolane and methanol, mixture of 1,3-dioxolane and water, to obtain the suspension comprising said crystalline Form of erlotinib hydrochloride,
wherein the mixture of 1,3-dioxolane and water has about 5 to about 10% v/v of water, the mixture of 1,3-dioxolane and methanol has about 20% to about 40% v/v of methanol, mixture of n-butanol and water has about 5% to about 10% v/v of water, the mixture of s-butanol and water has about 10% v/v of water and the mixture of methoxyethanol and water has about 10% v/v of water.

33. The process of claim 32, wherein the erlotinib hydrochloride is obtained by reacting erlotinib base with HCl in said solvent.

34. The process of claim 33, wherein said erlotinib base is dissolved in the solvent at about 20° C. to about 60° C. to form a solution.

35. The process of claim 34, wherein the HCl is added to said solution.

36. The process of any of claims 33-35, wherein said HCl is in a gas form or in a form of a solution.

37. The process of claim 36, wherein said HCl is in a form of a solution in an organic solvent.

38. The process of claim 37, wherein said organic solvent is ether, preferably diethylether.

39. The process of claim 36, wherein said HCl is in a form of an aqueous solution.

40. The process of claim 39, wherein concentration of said aqueous solution is about 30 to about 44% w/w.

41. The process of any of claims 34-40, wherein, prior to the addition of said HCl, the temperature of the solution is set to about 0° C. to about 60° C.

42. The process of any of claims 32-41, further comprising recovering said crystalline form of erlotinib hydrochloride exhibits an X-ray powder diffraction pattern having characteristics peaks at approximately 6.3, 7.8, 9.5, 12.5, 20.2 and 22.4±0.2 degrees 2-theta.

43. The process of claim 32, wherein the solvent is methanol, and wherein said process comprises (i) dissolving erlotinib HCl in methanol, and (ii) precipitating erlotinib hydrochloride exhibits an X-ray powder diffraction pattern having characteristics peaks at approximately 6.3, 7.8, 9.5, 12.5, 20.2 and 22.4±0.2 degrees 2-theta. More preferably XRD diffraction peak at about 6.3±0.2 degrees 2-theta.

44. The process of claim 43, wherein said dissolution is achieved at a temperature of about 65° C.

45. The process of any of claims 43-44, wherein said precipitating is done by cooling the solution to a temperature of about +100 to −10° C.

46. The process of any of claims 43-45, further comprising maintaining the suspension for about 15 hours to about 24 hours at a temperature of about −10° to about −40° C.

47. The process of any of claims 43-46, further comprising recovering said crystalline form of erlotinib hydrochloride exhibits an X-ray powder diffraction pattern having characteristics peaks at approximately 6.3, 7.8, 9.5, 12.5, 20.2 and 22.4±0.2 degrees 2-theta.

48. A process for preparing crystalline form of erlotinib hydrochloride exhibits an X-ray powder diffraction pattern having characteristics peaks at approximately 6.3, 7.8, 9.5, 12.5, 20.2 and 22.4±0.2 degrees 2-theta, comprising slurring crystalline form of erlotinib hydrochloride exhibits an X-ray powder diffraction pattern having characteristic peaks at approximately 5.7, 9.8, 10.1, 10.3, 18.9, 19.5, 21.3, 24.2, 26.2 and 29.2±0.2 degrees 2-theta in a solvent selected from a group consisting of: methanol, mixture of 1,3-dioxolane and water, n-heptane, and diethyl ether, wherein the mixture of 1,3-dioxolane and water has about 5 to about 10% v/v of water and mixtures thereof.

49. The process of claim 48, wherein said slurrying is done at a temperature of about 0° C. to about 30-C.

50. The process of any of claims 48-49, further comprising recovering said crystalline form of erlotinib hydrochloride exhibits an X-ray powder diffraction pattern having characteristics peaks at approximately 6.3, 7.8, 9.5, 12.5, 20.2 and 22.4±0.2 degrees 2-theta.
